(19) United States
(12) Patent Application Publication
(10) Pub. No.: US 2025/0032767 A1
(43) Pub. Date: Jan. 30, 2025

(54) HEMOSTATIC SPRINGS AND SEALS, AND METHODS

(71) Applicant: Medtronic, Inc., Minneapolis, MN (US)

(72) Inventor: Jake DUNLEA, Galway City (IE)

(73) Assignee: Medtronic, Inc., Minneapolis, MN (US)

(21) Appl. No.: 18/631,385

(22) Filed: Apr. 10, 2024

Related U.S. Application Data
(60) Provisional application No. 63/529,524, filed on Jul. 28, 2023.

(51) Int. Cl. A61M 39/06

(52) U.S. Cl. CPC A61M 39/0613; A61M 2039/062; A61M 2039/0633; A61M 2039/0673; A61M 2039/0686; A61M 2205/0216

(57) ABSTRACT

Hemostatic springs and hemostatic seals comprising hemostatic springs facilitate introducing a surgical device into the vasculature of a patient during a surgical procedure. Each hemostatic spring comprises a radial array of spring segments positioned on a circular path about a central axis. Each hemostatic seal is configured to reduce an axial length of the hemostatic spring to constrict the radial array of spring segments to reduce a cross-sectional area of the central passage. Methods include axially inserting the surgical device into the hemostatic seal while compression from the hemostatic seal minimizes fluid leakage. Methods can further include reducing a frictional force of inserting the device into the hemostatic seal by reducing the axial compression to dilate the hemostatic spring.

### CROSS-REFERENCE TO RELATED APPLICATIONS

This application claims the benefit of U.S. Provisional Patent Application Ser. No. 63/529,524, filed Jul. 28, 2023, the entire content of which is incorporated herein by reference.

### FIELD

The present disclosure relates generally to hemostatic springs, hemostatic seals, and methods and, more particularly, to hemostatic springs and hemostatic seals with hemostatic springs comprising a radial array of spring segments and methods of introducing a device into a vasculature of a patient by axially inserting the device into a hemostatic seal.

### BACKGROUND

Transcatheter techniques have been developed to provide minimally invasive procedures to introduce medical devices into a patient by passing the medical device through the vasculature of the patient to the target site. Typical introducer systems include an introducer sheath for introduction of a device by way of a transcatheter. The device is known to be passed through a hemostatic valve to prevent blood loss from the introducer sheath when inserting the medical device into the sheath. However, the known biasing mechanisms of conventional hemostatic valves can resist insertion of the device through the valve. The resistance can complicate the procedure and may damage the device or components of the delivery device.

### SUMMARY

The following presents a simplified summary of the disclosure to provide a basic understanding of some aspects described in the detailed description.

Features of the present disclosure provides hemostatic springs and seals that can facilitate dynamic changes of the forces being applied by the hemostatic spring while inserting the device through the hemostatic seal. Providing for dynamic changing of hemostatic spring force can provide the clinician with greater control of the surgical procedure to provide the correct balance of pressure to resist blood loss while minimizing resistance to device insertion through the hemostatic seal to reduce complications and prevent damage to the medical device or components of the delivery device.

In aspects, a hemostatic spring comprises a first end portion, a second end portion opposite the first end portion, and a radial array of spring segments positioned on a circular path about a central axis. Each spring segment of the radial array of spring segments comprises a first end, a second end, and a central location positioned between the first end and the second end. The first end of each spring segment is attached to the first end portion and each spring segment tapers toward the central location in a first direction of the central axis extending from the first end portion toward the second end portion. A second end of each spring segment is attached to the second end portion and each spring segment tapers toward the central location in a second direction of the central axis opposite the first direction. The central location of each spring segment is spaced on the circular path from the central location of each adjacent spring segment of the radial array of spring segments when a force is not applied to the radial array of spring segments.

In further aspects, a hemostatic seal comprises a hemostatic spring comprising a first end portion, a second end portion opposite the first end portion, and a radial array of spring segments positioned on a circular path about a central axis. Each spring segment of the radial array of spring segments comprises a first end, a second end, and a central location positioned between the first end and the second end. The first end of each spring segment is attached to the first end portion and each spring segment tapers toward the central location in a first direction of the central axis extending from the first end portion toward the second end portion. A second end of each spring segment is attached to the second end portion and each spring segment tapers toward the central location in a second direction of the central axis opposite the first direction. The central location of each spring segment is spaced on the circular path from the central location of each adjacent spring segment of the radial array of spring segments when a force is not applied to the radial array of spring segments. The hemostatic seal further comprises a proximal mount comprising a proximal aperture aligned with the central axis extending through a central passage of the hemostatic spring, wherein the first end portion of the hemostatic spring is mounted to the proximal mount. The hemostatic seal further comprises a distal mount comprising a distal aperture aligned with the central axis, wherein the second end portion of the hemostatic spring is mounted to the distal mount. The hemostatic seal is configured to reduce the distance between the proximal mount and the distal mount along the central axis to constrict the radial array of spring elements to reduce a cross-sectional area of the central passage.

In still further aspects, a method of introducing a device into a vasculature of a patient comprising percutaneously inserting an introducer sheath into the vasculature of the patient wherein the introducer sheath is provided with a hemostatic seal comprising a hemostatic spring under an axial compression to minimize leakage of fluid from the introducer sheath. The method further comprises axially inserting the device into the hemostatic seal, and reducing a frictional force of inserting the device into the hemostatic seal by reducing the axial compression to dilate the hemostatic spring. The method further comprises passing the device into the introducer sheath from the hemostatic seal and passing the device from the introducer sheath to the vasculature of the patient.

### BRIEF DESCRIPTION OF THE DRAWINGS

These and other features, aspects and advantages are better understood when the following detailed description is read with reference to the accompanying drawings, in which:

FIG. 1 is a schematic view of a hemostatic seal in accordance with aspects of the disclosure being used during a surgical procedure;

FIG. 2 is a schematic cross-sectional view of the hemostatic seal of FIG. 1;

FIG. 3 is a schematic cross-sectional view of the hemostatic seal of FIG. 2 with a surgical device being inserted through the hemostatic seal;

FIG. 4 is a side view of a hemostatic spring in accordance with embodiments of the disclosure when an axial compressive force is not applied compress the hemostatic spring;

FIG. 5 is an end view of the hemostatic spring along view 5-5 of FIG. 4;

FIG. 6 is a side view of the hemostatic spring of FIG. 4 when an axial compressive force is applied to compress the hemostatic spring;

FIG. 7 is an end view of the hemostatic spring along view 7-7 of FIG. 6;

FIG. 8 is a side view of a hemostatic spring in accordance with further embodiments of the disclosure when an axial compressive force is applied to compress the hemostatic spring;

FIG. 9 is a side view of the hemostatic spring of FIG. 8 when an axial compressive force is not applied to compress the hemostatic spring;

FIG. 10 is a side schematic view representing hemostatic springs of further embodiments of the disclosure when an axial compressive force is applied to compress the hemostatic springs;

FIG. 11 is a side view of one embodiment of the hemostatic spring of FIG. 10 when an axial compressive force is not applied to compress the hemostatic spring;

FIG. 12 is a side view of another embodiment of the hemostatic spring of FIG. 10 when an axial compressive force is not applied to compress the hemostatic spring;

FIG. 13 is a schematic cross-sectional view of a hemostatic seal in accordance with embodiments of the disclosure;

FIGS. 14-17 illustrate introducing a surgical device through the hemostatic seal of FIG. 13 during a surgical procedure;

FIG. 18 is a schematic cross-sectional view of a hemostatic seal in accordance with embodiments of the disclosure;

FIG. 19 illustrates introducing a surgical device through the hemostatic seal of FIG. 18 during a surgical procedure;

FIG. 20 is a schematic cross-sectional view of a hemostatic seal in accordance with embodiments of the disclosure; and

FIG. 21 illustrates introducing a surgical device through the hemostatic seal of FIG. 20 during a surgical procedure.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

The terms “distal” and “proximal” are used in the following description with respect to a position or direction relative to an expected position of a treating clinician. “Distal” or “distally” are a position distant from or in a direction away from the clinician. “Proximal” and “proximally” are a position near or in a direction toward the clinician. In addition, as used herein, the terms “outward” or “outwardly” refer to a position radially away from a central axis of the hemostatic spring and the terms “inward” or “inwardly” refer to a position radially toward a central axis of the hemostatic spring.

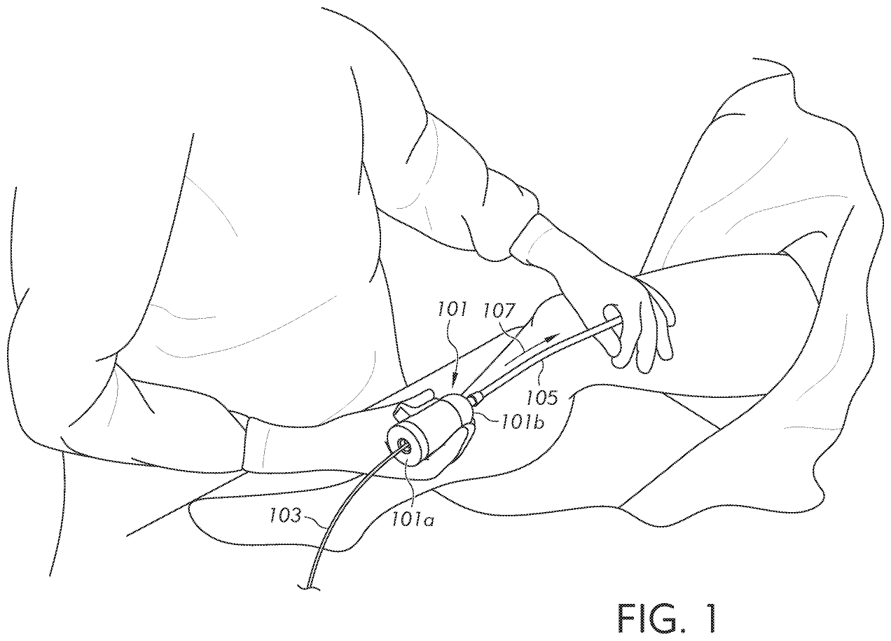

FIG. 1 is a schematic view of a hemostatic seal 101 in accordance with aspects of the disclosure being used during a surgical procedure to insert a surgical device into the vasculature of a patient with a catheter 103. As shown, a catheter is inserted in a distal direction 107 into a proximal end 101a of the hemostatic seal 101 while an introducer sheath 105 extends in the distal direction 107 from a distal end 101b of the hemostatic seal 101. The distal end of the introducer sheath 105 can be inserted into the vasculature of a patient where the surgical device is to be introduced.

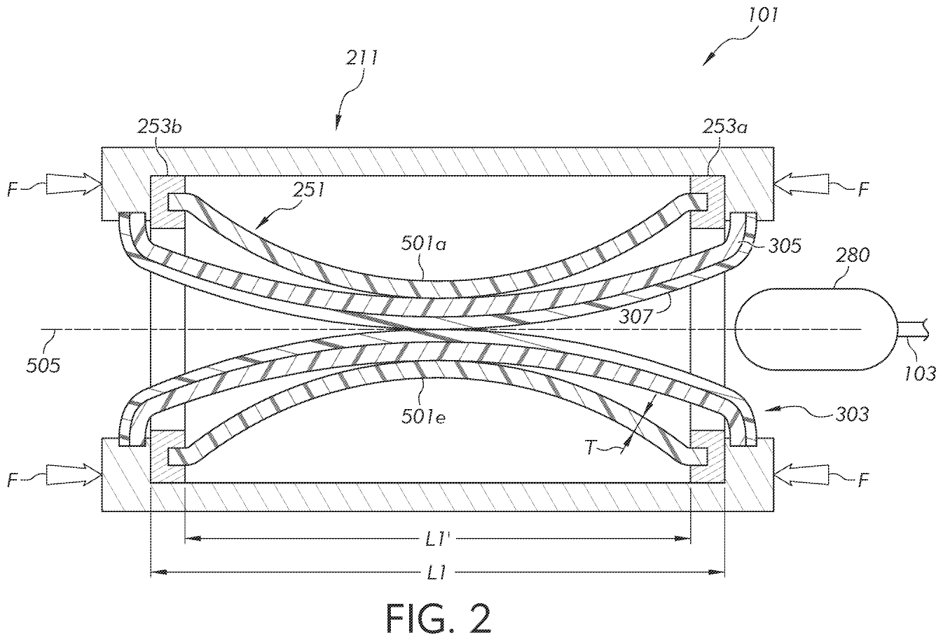

FIG. 2 illustrates a schematic cross-sectional view of an embodiment of the hemostatic seal 101 of FIG. 1 comprising a housing 211 containing a hemostatic spring 251. The hemostatic spring 251 comprises a first end portion 253a and a second end portion 253b opposite the first end portion 253a. As shown in FIGS. 5 and 7, the hemostatic spring 251 comprises a radial array of spring segments 501a-h positioned on a circular path 503 about a central axis 505 of the hemostatic spring 251. As shown, the spring segments 501a-h can be equally spaced from one another about the circular path 503 to equalize the resultant force applied by the spring segments in axial compression. Eight spring segments are illustrated although any plurality of spring segments may be provided in further embodiments.

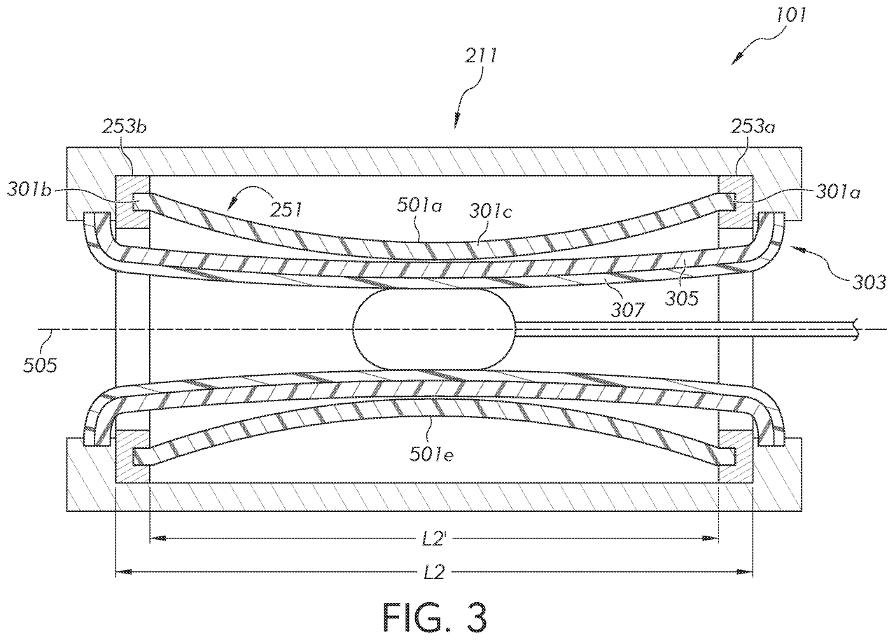

In some embodiments, as shown, the spring segments 501a-h can be substantially identical to one another. As such, the discussion of any spring segment can equally apply to all spring segments. As shown in FIG. 3, each spring segment 501a-h of the radial array of spring segments 501a-h comprises a first end 301a, a second end 301b, and a central location 301c positioned between the first end 301a and the second end 301b. In some embodiments, as shown in FIG. 3, the first end 301a of each spring segment 501a-h is attached to the first end portion 253a and a second end 301b of each spring segment 501a-h is attached to the second end portion 253b of the hemostatic spring 251.

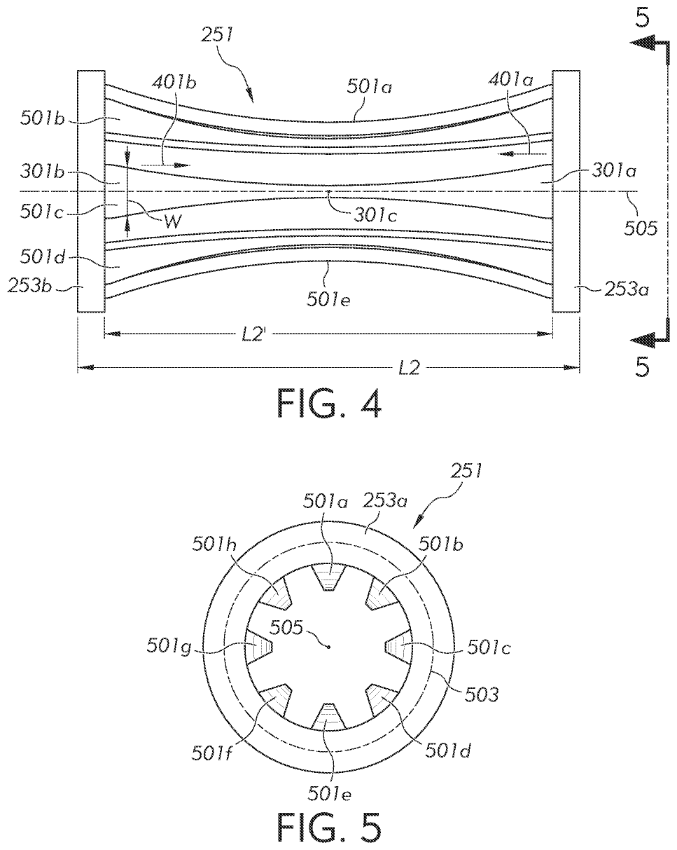

Each spring segment 501a-h of the radial array of spring segments 501a-h may taper in a direction from each end 301a, 301b toward the central location 301c of the spring segment. For example, with reference to spring segment 501c in FIG. 4, each spring segment 501a-h tapers toward the central location 301c in a first direction 401a of the central axis 505 extending from the first end portion 253a toward the second end portion 253b, and tapers toward the central location 301c in a second direction 401b of the central axis 505 opposite the first direction 401a. In some embodiments, the tapering of the spring segments 501a-h can be continuously tapering or discontinuously tapering (e.g., periodically, or intermittently tapering). In the illustrated embodiment of FIGS. 4-7, each spring segment spring segment 501a-h continuously tapers toward the central location 301c in the first direction 401a and the second direction 401b. Still further, in some embodiments, the tapering may begin at a location spaced from the corresponding end portion or may begin at the end portion. In the illustrated embodiment of FIGS. 4-7, the tapering begins at the end portion and continuously tapers until it reaches the central location 301c (e.g., the midpoint of the spring segment in the embodiment of FIGS. 4-7).

Throughout the disclosure, as shown in FIG. 2, the length “L1” is the overall length of the fully compressed hemostatic spring 251 while the length L1′ is the length of a spring segment of the fully compressed hemostatic spring 251 that is considered the distance extending in the direction of the central axis 505 between the first end portion 253a and the second end portion 253b. As shown in FIG. 3, the length “L2” is the overall length of an uncompressed hemostatic spring 251 while the length L2′ is the length of a spring segment of an uncompressed hemostatic spring 251 that is considered the distance extending in the direction of the central axis 505 between the first end portion 253a and the second end portion 253b. The thickness “T” of the spring segment is considered the cross-sectional thickness of the spring segment along a cross-sectional plane including the central axis 505 of the hemostatic spring 251 and the central axis of the spring segment. The width “W” of the spring segment is considered the distance across the spring segment in a width direction that is perpendicular to the direction of the thickness “T” and perpendicular to a direction of the central axis 505.

Unless otherwise stated, for purposes of this disclosure, tapering of the spring segments throughout the disclosure means tapering of the width “W” of the spring segment and/or tapering of the thickness “T” of the spring segment. For example, as shown, in FIG. 4, the tapering of the spring segments 501a-h can comprise tapering of the width “W” while as shown in FIG. 2, the thickness “T” may not be tapered although the thickness may be tapered (in addition or alternative to the width) in further embodiments.

As shown in FIG. 3, the first end 301a and the second end 301b of the spring segments 501a-h may each be received in a corresponding mounting socket of the first and second end portions 253a, 253b. In some embodiments the first and second ends 301a, 301b can be permanently fixedly attached within the mounting sockets, e.g., by interference fit, welding, or other fastening technique. In further embodiments, the spring segments and end portions may be formed as a single monolithic member, for example, by injection molding.

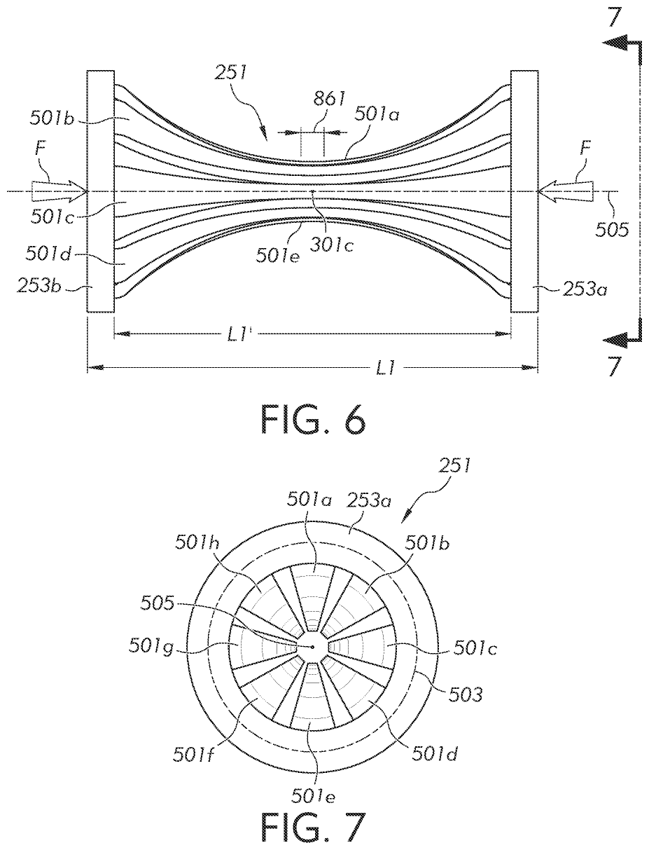

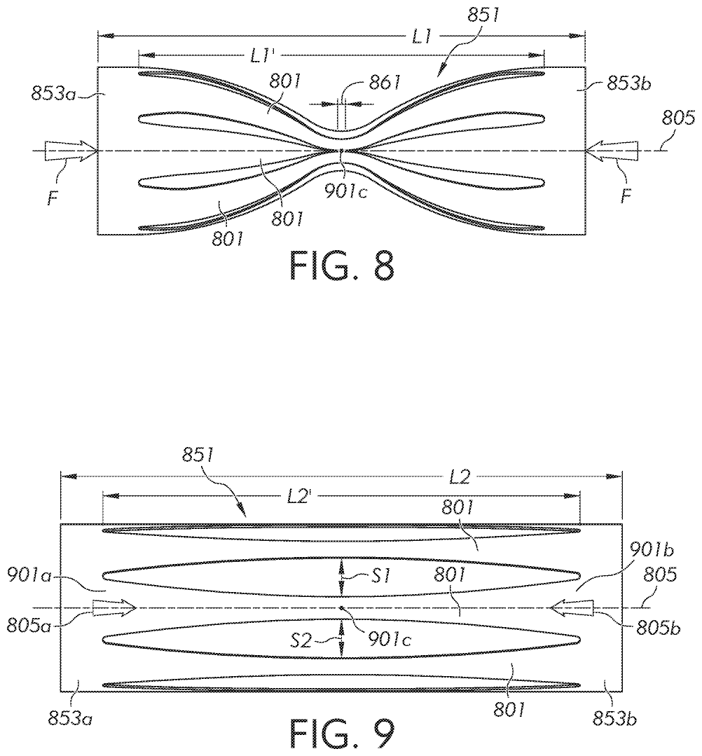

The hemostatic spring can be formed from a wide range of flexible, resilient materials such as polymeric material, spring steel, stainless steel. Furthermore, as shown in the embodiment of FIGS. 2-7, the hemostatic spring 251 can comprise multiple components that are assembled together. However, as mentioned previously in alternative embodiments, the hemostatic spring can comprise a single monolithic member. For example, FIGS. 8-9 disclose another embodiment of a hemostatic spring 851 fabricated from a single one-piece tube. As shown in FIG. 8, the hemostatic spring 851 is configured to be axially compressed to constrict the radial array of spring segments 801 to a minimum cross-sectional area along a midpoint length 861 centered at the midpoint comprising a central location 901c of the spring segments. Likewise, as shown in FIG. 6, the hemostatic spring 251 is configured to be axially compressed to constrict the radial array of spring segments 501a-h to a minimum cross-sectional area along a midpoint length 861 centered at the midpoint comprising the central location 301c of the spring segments.

For purposes of this application, the midpoint length can be experimentally determined by placing the hemostatic spring between two parallel steel plates wherein the central axis of the hemostatic spring is perpendicular to inner contact surfaces of the parallel steel plates. The steel plates are moved together in 0.5 millimeter (mm) increments to incrementally constrict the radial array of spring elements to reduce a cross-sectional area of the central passage. Lengths of the central portion having the minimum cross-sectional area of the central passage is measured after each incremental adjustment of the steel plates. The midpoint length 861 of the hemostatic spring is the length of the minimum cross-sectional area of the central passage at the increment prior to the incremental adjustment that causes failure of the hemostatic spring by plastic deformation or breaking of the hemostatic spring. In some embodiments, the midpoint length 861 of the hemostatic spring can be within a range of from about 1% to about 5% of the length L1′ of each identical spring segment in the radial array of spring segments.

As shown in FIGS. 8-9, the hemostatic spring 851 comprises many features that are similar to the features of the hemostatic spring 251 of FIGS. 4-7. The hemostatic spring 851 comprises a first end portion 853a and a second end portion 853b opposite the first end portion 853a. The hemostatic spring 851 comprises a radial array of spring segments 801 positioned on a circular path about a central axis 805 of the hemostatic spring 851. As shown, the spring segments 801 can be equally spaced from one another about the circular path to equalize the resultant force applied by the spring segments in axial compression. As can be appreciated, eight spring segments are provided although any plurality of spring segments may be provided in further embodiments.

In some embodiments, as shown, the spring segments 801 can be substantially identical to one another. As such, the discussion of any spring segment will equally apply to all spring segments. As shown in FIG. 9, each spring segment 801 of the radial array of spring segments 801 comprises a first end 901a, a second end 901b, and a central location 901c positioned between the first end 901a and the second end 901b. In some embodiments, as shown in FIG. 9, the first end 901a of each spring segment 801 is attached to the first end portion 853a and a second end 901b of each spring segment 801 is attached to the second end portion 853b of the hemostatic spring 851.

Each spring segment 801 of the radial array of spring segments 801 may taper in a direction from each end portion 853a, 853b toward the central location 901c of the spring segment. For example, with reference to FIG. 9, each spring segment 801 tapers toward the central location 901c in a first direction 805a of the central axis 805 extending from the first end portion 853a toward the second end portion 853b, and tapers toward the central location 901c in a second direction 805b of the central axis 805 opposite the first direction 805a. In some embodiments, the tapering of the spring segments 801 can be continuously tapering or discontinuously tapering (e.g., periodically, or intermittently tapering). In the illustrated embodiment of FIGS. 8-9, each spring segment spring segment 801 continuously tapers toward the central location 901c in a first direction 805a of the central axis 805 extending from the first end portion toward 853a the second end portion 853b, and continuously tapers toward the central location 901c in a second direction 805b of the central axis 805 opposite the first direction 805a. Still further, in some embodiments, the tapering may begin at a location spaced from the corresponding end portion or may begin at the end portion. In the illustrated embodiment of FIGS. 8-9, the tapering begins at the end portion and continuously tapers until it reaches the central location 901c (e.g., the midpoint of the spring segment in the embodiment of FIGS. 8-9).

As shown in FIGS. 8-9, in some embodiments, the hemostatic spring 851 is fabricated from a single one-piece tube. As shown in FIG. 8, the hemostatic spring 851 is configured to be axially compressed to constrict the radial array of spring segments 801 to a minimum cross-sectional area along the midpoint length 861 centered at the midpoint comprising the central location 901c of the spring segments. Likewise, as shown in FIG. 6, the hemostatic spring 251 is configured to be axially compressed to constrict the radial array of spring segments 501a-h to a minimum cross-sectional area along a midpoint length 861 centered at the midpoint comprising the central location 301c of the spring segments.

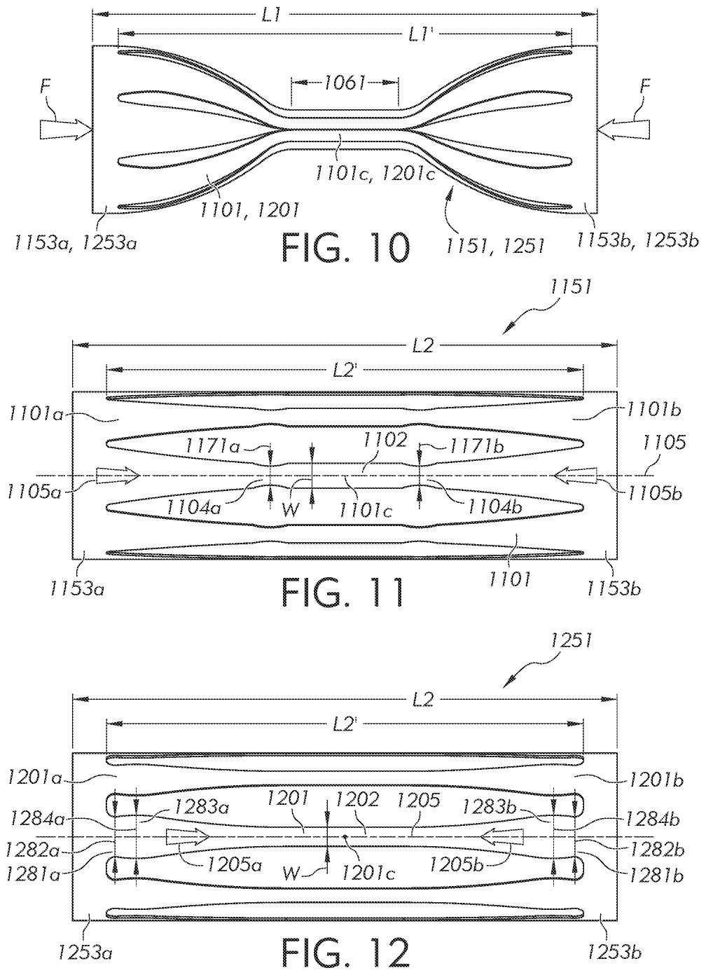

FIGS. 11-12 disclose alternative embodiments of hemostatic springs 1151, 1251 that can each be fabricated from a single one-piece tube. FIG. 10 is a schematic representation of the hemostatic springs 1151, 1251 that are axially compressed to constrict the radially array of spring segments 1101, 1201 to a minimum cross-sectional area along a central segment length 1061 of the central segment comprising the central location 1101c, 1201c of the spring segments 1101, 1201.

For purposes of this application, the central segment length 1061 can be experimentally determined by placing the hemostatic spring between two parallel steel plates wherein the central axis of the hemostatic spring is perpendicular to inner contact surfaces of the parallel steel plates. The steel plates are moved together in 0.5 millimeter (mm) increments to incrementally constrict the radial array of spring elements to reduce a cross-sectional area of the central passage. Lengths of the central portion having the minimum cross-sectional area of the central passage is measured after each incremental adjustment of the steel plates. The central segment length 1061 of the hemostatic spring is the length of the minimum cross-sectional area of the central passage at the increment prior to the incremental adjustment that causes failure of the hemostatic spring by plastic deformation or breaking of the hemostatic spring. In some embodiments, the central segment length 1061 of the hemostatic spring can be within a range of from greater than 5% to less than 50% of the length L1′ of each identical spring segment in the radial array of spring segments.

As shown in FIG. 11, the hemostatic spring 1151 a first end portion 1153a and a second end portion 1153b opposite the first end portion 1153a. The hemostatic spring 1151 comprises a radial array of spring segments 1101 positioned on a circular path about a central axis 1105 of the hemostatic spring 1151. As shown, the spring segments 1101 can be equally spaced from one another about the circular path to equalize the resultant force applied by the spring segments in axial compression. As can be appreciated, eight spring segments are provided although any plurality of spring segments may be provided in further embodiments.

In some embodiments, as shown, the spring segments 1101 can be substantially identical to one another. As such, the discussion of any spring segment will equally apply to all spring segments. As shown in FIG. 11, each spring segment 1101 of the radial array of spring segments 1101 comprises a first end 1101a, a second end 1101b, and a central location 1101c positioned between the first end 1101a and the second end 1101b. In some embodiments, the first end 1101a of each spring segment 1101 is attached to the first end portion 1153a and a second end 1101b of each spring segment 1101 is attached to the second end portion 1153b of the hemostatic spring 1151.

Each spring segment 1101 of the radial array of spring segments 1101 may taper in a direction from each end portion 1153a, 1153b toward the central location 1101c of the spring segment. For example, as shown, each spring segment 1101 tapers toward the central location 1101c in a first direction 1105a of the central axis 1105 extending from the first end portion 1153a toward the second end portion 1153b, and tapers toward the central location 1101c in a second direction 1105b of the central axis 1105 opposite the first direction 1105a. In some embodiments, the tapering of the spring segments 1101 can be continuously tapering or discontinuously tapering (e.g., periodically, or intermittently tapering). In the illustrated embodiment, each spring segment spring segment 1101 continuously tapers toward the central location 1101c in the first direction 1105a of the central axis 1105, and continuously tapers toward the central location 1101c in a second direction 1105b of the central axis 1105. Still further, in some embodiments, the tapering may begin at a location spaced from the corresponding end portion or may begin at the end portion. In the illustrated embodiment of FIG. 11, the tapering begins at the end portion and continuously tapers until it reaches the central location 1101c.

As further illustrated in FIG. 11, the central location 1101c of each spring segment 1101 can comprise a central segment 1102. In some embodiments, the central segment 1102 can comprise a width “W” that is substantially constant along the central axis 1105. The substantially constant width “W” can provide a central portion with a consistent cross-sectional passage dimension along the central segment length 1061 to provide consistent sealing against the surgical device passing through the hemostatic seal. Each spring segment 1101 continuously tapers from the first end 1101a to a first central waist 1104a of the central location 1101c, and continuously tapers from the second end 1101b to a second central waist 1104b of the central location 1101c. As shown, the central location 1101c therefore comprises the central segment 1102, the first central waist 1104a, and the second central waist 1104b, wherein the central segment 1102 is positioned between the first central waist 1104a and the second central waist 1104b. Each spring segment 1101 can continuously taper from the first end 1101a to the first central waist 1104a of the central location 1101c, and continuously taper from the second end 1101b to the second central waist 1104b of the central location 1101c, wherein the central segment 1102 is positioned between the first central waist 1104a and the second central waist 1104b. As further illustrated, a width “W” of the central segment can be greater than a width 1171a of the first central waist 1104a and a width 1171b of the second central waist 1104b. Providing the reduced widths at the first and second central waists 1104a, 1104b can encourage pivoting of the spring segments 1101 when constricting the spring segments 1101 at these locations to achieve the configuration shown in FIG. 10 while reducing stress concentrations at the pivot points.

FIG. 12 illustrates another embodiment of the hemostatic spring 1251 schematically illustrated in FIG. 10. As shown, the hemostatic spring 1251 comprises a first end portion 1253a and a second end portion 1253b opposite the first end portion 1253a. The hemostatic spring 1251 comprises a radial array of spring segments 1201 positioned on a circular path about a central axis 1205 of the hemostatic spring 1251. As shown, the spring segments 1201 can be equally spaced from one another about the circular path to equalize the resultant force applied by the spring segments in axial compression. As can be appreciated, eight spring segments are provided although any plurality of spring segments may be provided in further embodiments.

In some embodiments, as shown, the spring segments 1201 can be substantially identical to one another. As such, the discussion of any spring segment will equally apply to all spring segments. As shown in FIG. 12, each spring segment 1201 of the radial array of spring segments 1201 comprises a first end 1201a, a second end 1201b, and a central location 1201c positioned between the first end 1201a and the second end 1201b. In some embodiments, the first end 1201a of each spring segment 1201 is attached to the first end portion 1253a and a second end 1201b of each spring segment 1201 is attached to the second end portion 1253b of the hemostatic spring 1251.

Each spring segment 1201 of the radial array of spring segments 1201 may taper in a direction from each end portion 1253a, 1253b toward the central location 1201c of the spring segment. For example, as shown, each spring segment 1201 tapers toward the central location 1201c in a first direction 1205a of the central axis 1205 extending from the first end portion 1253a toward the second end portion 1253b, and tapers toward the central location 1201c in a second direction 1205b of the central axis 1205 opposite the first direction 1205a. In some embodiments, the tapering of the spring segments 1201 can be continuously tapering or discontinuously tapering (e.g., periodically, or intermittently tapering). In the illustrated embodiment, each spring segment spring segment 1201 continuously tapers toward the central location 1201c in the first direction 1205a of the central axis 1205, and continuously tapers toward the central location 1201c in a second direction 1205b of the central axis 1205. Still further, as shown, the tapering can begin at a location spaced from the corresponding end portion or may begin at the end portion. For example, as shown in FIG. 12, the first end 1201a of each spring segment 1201 is attached at a first end waist 1281a to the first end portion 1253a and the second end 1201b of each spring segment 1201 is attached at a second end waist 1281b to the second end portion 1253b. Each spring segment 1201 tapers (e.g., continuously tapers) in the first direction 1205a from a first intermediate portion 1283a toward the central location 1201c wherein the central location 1201c can comprise a central segment in some embodiments. Each spring segment 1201 further tapers (e.g., continuously tapers) in the second direction 1205b of the central axis 1205 from a second intermediate portion 1283b toward the central location 1201c wherein the central location 1201c can comprise a central segment in some embodiments.

As further illustrated in FIG. 12, the first end waist 1281a is positioned between the first end portion 1253a and the first intermediate portion 1283a. The second end waist 1281b is positioned between the second end portion 1253b and the second intermediate portion 1283b. As shown, the first intermediate portion 1283a comprises a maximum width 1284a that is greater than a minimum width 1282a of the first end waist 1281a, and the second intermediate portion 1283b comprises a maximum width 1284b greater than a minimum width 1282b of the second end waist 1281b. In some embodiments, as shown, the minimum width 1282a of the first end waist 1281a and the minimum width 1282b of the second end waist 1281b can each be greater than a minimum width “W” of the central location 1201c (e.g., central segment). Furthermore, as shown, in some embodiments, the central location 1201c of each spring segment 1201 can comprise a central segment 1202 of the corresponding spring segment with a substantially constant width “W” along the central axis 1205. Providing the end waists with reduced minimum width relative to the maximum width of the associated intermediate portion helps reduce stress and plastic deformation of the hemostatic spring when the hemostatic spring is axially compressed to the configuration schematically illustrated in FIG. 10.

As shown in FIGS. 4-5, 9, and 11-12, each embodiment of the hemostatic spring 251, 851, 1151, 1251 provides that the central location 301c, 901c, 1101c, 1201c of each spring segment 501a-h, 801, 1101, 1201 is spaced on the circular path 503 from the central location 301c, 901c, 1101c, 1201c of each adjacent spring segment of the radial array of spring segments 501a-h, 801, 1101, 1201 when a force is not applied to the radial array of spring segments 501a-h, 801, 1101, 1201. For example, as shown in FIG. 9, when the axial forces are not present that compress the hemostatic spring 851, a space “S1” exists on the circular path between the spring segment 801 (i.e., the central spring segment as it appears in FIG. 9) and the adjacent spring segment 801 positioned above the central spring segment as it appears in FIG. 9. Further, when axial forces are not present that compress the hemostatic spring 851, as further shown in FIG. 9, a space “S2” exists on the circular path between the central spring segment 801 and the spring segment 801 positioned below the central spring segment as it appears in FIG. 9. Therefore, in a relaxed state, the central locations of the spring segments are spaced apart from one another. In contrast, under axial compression (see opposed forces “F” in FIGS. 2, 6, and 8), the central locations constrict to radially contract toward the central axis of the hemostatic spring wherein the spring segments act together to narrow the passage passing through the hemostatic spring. While the spring segments are radially constricted, they also act to radially bias the hemostatic spring back to the original relaxed state. As such, once the compressive axial force “F” is removed, each of the central locations radially retract to dilate the passage and thereby return the hemostatic spring back to its original relaxed state.

As mentioned previously, in some embodiments, the hemostatic springs (e.g., hemostatic springs 851, 1151, 1251) can be fabricated as a one piece spring. For example, in some embodiments, the hemostatic spring can be fabricated from a tube that can then be laser cut or otherwise machined into the desired configuration (e.g., as shown in FIGS. 8-12). In such example, the first end portion 853a, 1153a, 1253a, and the second end portion 853b, 1153b, 1253b can each comprise a continuous circular cylindrical tube. A thickness of the first end portion, a thickness of the second end portion, and a thickness of each spring segment can also be approximately equal. Furthermore, each spring segment of the radial array of spring segments can be substantially straight and extend parallel to the central axis when a force is not applied to the radial array of spring segments. Although not shown, in some embodiments, a slight crimp may be provided at the center of the spring segments to help initiate inward rather than outward buckling of the spring segments when applying the axial force to the hemostatic spring.

Turning back to FIG. 3, in some embodiments, the hemostatic springs of the present disclosure may comprise one or more internal sleeves 303 that can line an internal passage of the hemostatic spring to help contain fluid and reduce friction between the surgical device and the hemostatic spring. In some embodiments, the internal sleeve 303 of hemostatic spring can comprise an elastomeric sublayer 305 (e.g., silicone) that may provide compliance to seal around the surgical device. In some embodiments, the internal sleeve 303 can further comprise a low friction polymer 307 (e.g., PTFE) to reduce friction and thereby ease passage of the device and reduce the likelihood of snagging on the sleeve.

Embodiments of hemostatic seals are now described that can incorporate any one of the hemostatic springs described throughout this application including the hemostatic springs 251, 851, 1151, 1251. For purposes of description of the embodiments of the hemostatic seals, the description of the various embodiments of the hemostatic seals will be discussed incorporating the hemostatic spring 851 illustrated in FIGS. 8-9 with the understanding that the description would similarly or identically apply to all hemostatic seals incorporating any of the other hemostatic springs (e.g., hemostatic springs 251, 1151, 1251).

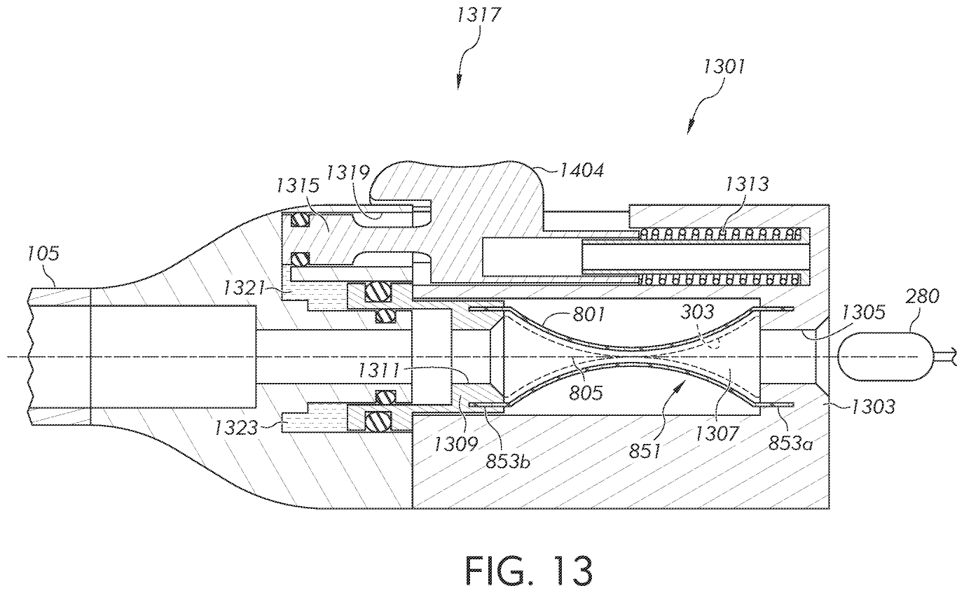

FIG. 13 illustrates one embodiment of a hemostatic seal 1301 comprising the hemostatic spring 851 described above although any hemostatic spring may be provided in accordance with embodiments of the disclosure. The hemostatic seal 1301 comprises a proximal mount 1303 comprising a proximal aperture 1305 aligned with the central axis 805 extending through a central passage 1307 of the hemostatic spring 851, wherein the first end portion 853a of the hemostatic spring 851 is mounted to the proximal mount 1303. The hemostatic seal 1301 can further comprise a distal mount 1309 comprising a distal aperture 1311 aligned with the central axis 805, wherein the second end portion 853b of the hemostatic spring 851 is mounted to the distal mount 1309.

The hemostatic seal 1301 is configured to reduce the distance between the proximal mount 1303 and the distal mount 1309 along the central axis 805 to constrict the radial array of spring elements 801 to reduce a cross-sectional area of the central passage 1307. As shown in FIG. 13, in one embodiment, a compression spring 1313 is configured to bias at least one of the proximal mount 1303 and the distal mount 1309 to reduce the distance between the proximal mount 1303 and the distal mount 1309 along the central axis 805 to constrict the radial array of spring elements 801. For example, as shown, the compression spring 1313 biases a linear piston 1315 of an actuator 1317 to the left within the hydraulic cylinder 1319 in the orientation shown in FIG. 13 to expel the hydraulic fluid 1321 from the hydraulic cylinder 1319 to enter a compression chamber 1323. The hydraulic fluid 1321 is therefore hydraulically compressed to apply force to the distal mount 1309 and therefore bias the distal mount 1309 reduce the distance between the proximal mount 1303 and the distal mount 1309 along the central axis 805 to constrict the radial array of spring elements 801.

The actuator 1317 of the hemostatic seal 1301 is also configured to act against the bias of the compression spring 1313 to selectively dilate the radial array of spring segments 801 to increase a cross-sectional area of the central passage 1307. In some embodiments, the actuator can comprise the illustrated trigger 1404.

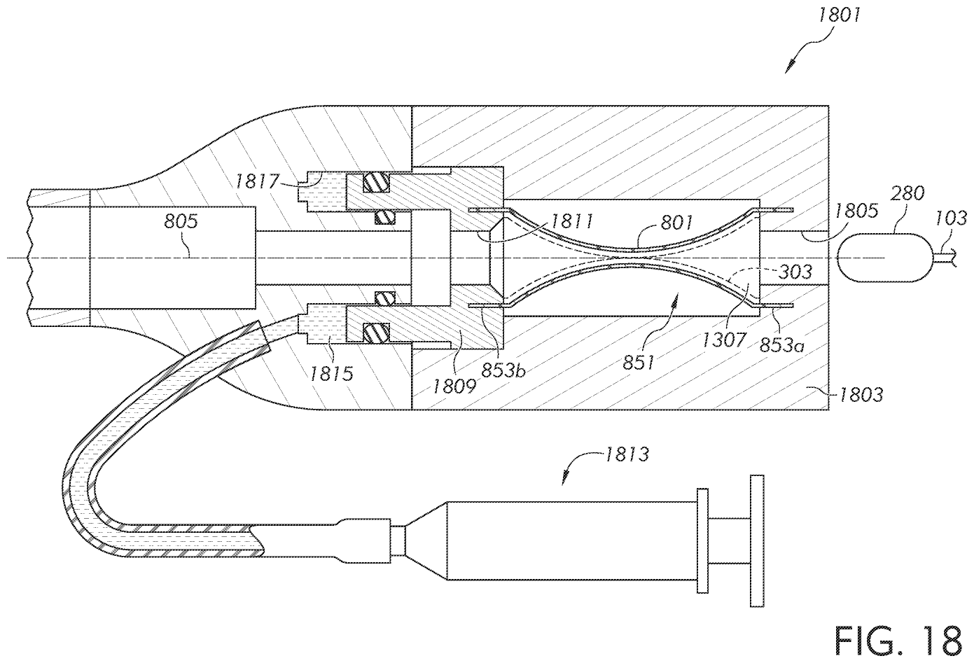

FIG. 18 illustrates another embodiment of a hemostatic seal 1801 comprising the hemostatic spring 851 described above although any hemostatic spring may be provided in accordance with embodiments of the disclosure. The hemostatic seal 1801 comprises a proximal mount 1803 comprising a proximal aperture 1805 aligned with the central axis 805 extending through a central passage 1307 of the hemostatic spring 851, wherein the first end portion 853a of the hemostatic spring 851 is mounted to the proximal mount 1803. The hemostatic seal 1801 can further comprise a distal mount 1809 comprising a distal aperture 1811 aligned with the central axis 805, wherein the second end portion 853b of the hemostatic spring 851 is mounted to the distal mount 1809.

The hemostatic seal 1801 is configured to reduce the distance between the proximal mount 1803 and the distal mount 1809 along the central axis 805 to constrict the radial array of spring elements 801 to reduce a cross-sectional area of the central passage 1307 to minimize leakage of fluid from the introducer sheath. As shown in FIG. 18, in one embodiment, a syringe 1813 is used to control hydraulic fluid 1815 within a pressure chamber 1817. As shown, the syringe 1813 can be positioned to pressurize the hydraulic fluid 1815 in the pressure chamber 1817 to bias the distal mount 1809 to reduce the distance between the proximal mount 1803 and the distal mount 1809 along the central axis 805 to constrict the radial array of spring segments 801. Although not shown, a lock may be provided on the syringe to releasably lock the syringe at a desired adjusted position wherein the clinician can later release it.

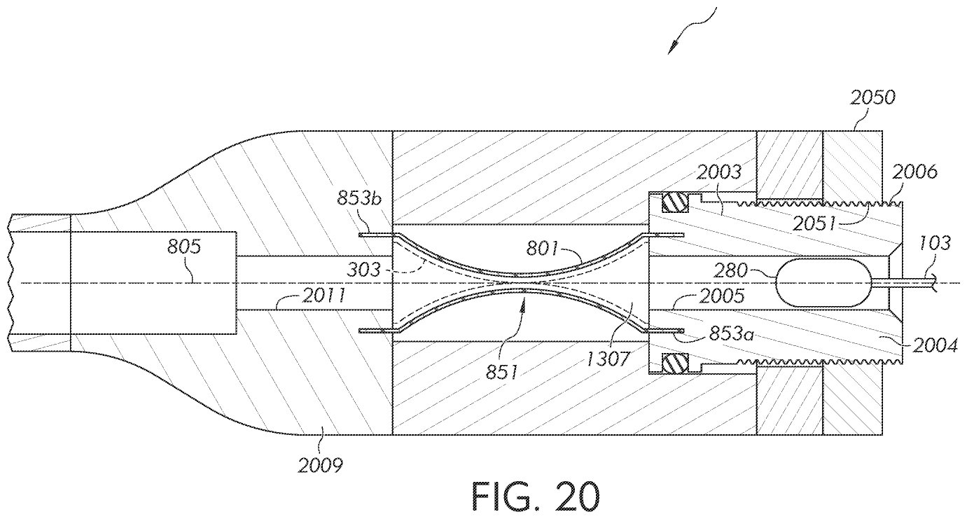

FIG. 20 illustrates another embodiment of a hemostatic seal 2001 comprising the hemostatic spring 851 described above although any hemostatic spring may be provided in accordance with embodiments of the disclosure. The hemostatic seal 2001 comprises a proximal mount 2003 comprising a proximal aperture 2005 aligned with the central axis 805 extending through a central passage 1307 of the hemostatic spring 851, wherein the first end portion 853a of the hemostatic spring 851 is mounted to the proximal mount 2003. The hemostatic seal 2001 can further comprise a distal mount 2009 comprising a distal aperture 2011 aligned with the central axis 805, wherein the second end portion 853b of the hemostatic spring 851 is mounted to the distal mount 2009.

The hemostatic seal 2001 is configured to reduce the distance between the proximal mount 2003 and the distal mount 2009 along the central axis 805 to constrict the radial array of spring elements 801 to reduce a cross-sectional area of the central passage 1307. As shown in FIG. 20, in one embodiment, a drive nut 2050 can comprise internal threads 2051 threadedly engaging external threads 2006 of drive shaft 2004 of the proximal mount 2003. Rotation of the drive nut 2050 about the central axis 805 can bias the proximal mount 2003 to reduce the distance between the proximal mount 2003 and the distal mount 2009 along the central axis 805 to constrict the radial array of spring segments 801.

As shown, in each of the hemostatic seals 1301, 1801, 2001, the proximal mount 1303, 1803, 2003 and distal mount 1309, 1809, 2009 may be provided with a circular ring reception aperture configured to snugly receive the tubular ends of the first and second end portions 853a, 853b of the tubular members forming the hemostatic spring 851. In some embodiments, the circular ring reception aperture may be deep enough to receive the entire end portions such that, once the end portions are fully inserted into the ring reception apertures, the distance between the proximal and distal mounts comprises the length of the spring segments. The circular ring reception apertures can be formed by boring, electrical discharge machining (EDM) or other techniques. Furthermore, the first and second end portions 853a, 853b may be welded, press-fit, adhered or otherwise attached to the circular ring reception apertures. In some embodiments, the first and second end portions 853a, 853b may be snuggly received within the circular ring reception apertures without being fixedly attached. Such embodiments may be desired in applications where the hemostatic spring will always be in compression such that attachment is not necessary. Avoiding attachment may be beneficial to permit replacing a damaged hemostatic spring or replacing a hemostatic spring with another hemostatic spring with a different spring constant that is more appropriate for the surgical device that will be used during a particular surgical procedure.

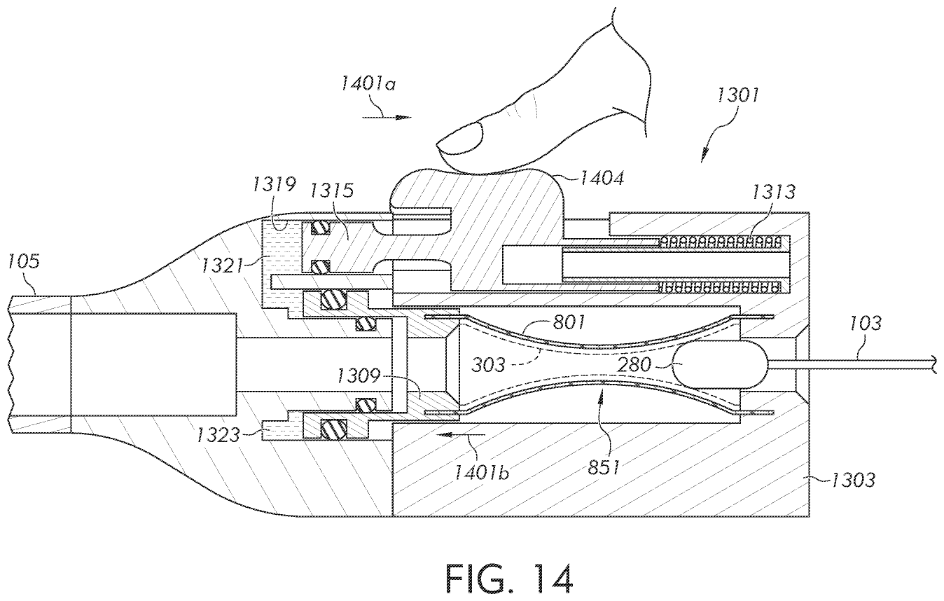

Methods of introducing a device 280 at the end of a catheter 103 into a vasculature of a patient with a hemostatic seal 101 will now be discussed with initial reference to FIGS. 1-2 and 13 with the understanding that similar or identical methods may be provided in the other embodiments of the disclosure. The method can begin by percutaneously inserting the introducer sheath 105 into the vasculature of the patient wherein the introducer sheath 105 is in communication with the central passage 1307 of the hemostatic spring 851 and the hemostatic spring 851 is in axial compression to minimize leakage of fluid from the introducer sheath 105. As shown in FIG. 14, the method further comprises axially inserting the device 280 into the hemostatic seal 1301. As further shown in FIG. 14, a clinician can begin reducing a frictional force of inserting the device into the hemostatic seal 1301 by reducing the axial compression to dilate the hemostatic spring. For example, the clinician may begin applying a force in direction 1401a to the trigger 1404 against the bias of compression spring 1313. As a result, the trigger 1404 shifts in the direction 1401a to compress the compression spring 1313 wherein the hydraulic fluid 1321 begins leaving the compression chamber 1323 and enters the hydraulic cylinder 1319. The compression chamber 1323 therefore begins to decompress wherein the distal mount 1309 begins moving in direction 1401b. Consequently, as the distal mount 1309 is moving apart from the proximal mount 1303, the hemostatic spring 851 slightly dilates. Thus, the clinician can reduce the friction of inserting the device 280 into the hemostatic seal 1301 while still maintaining a proper sealed interface between the hemostatic seal 1301 and the device 280.

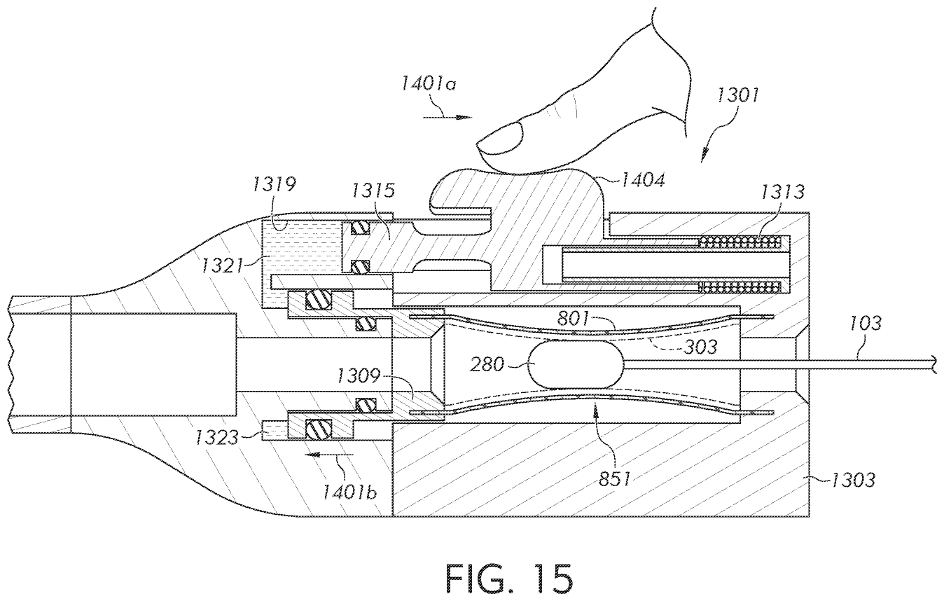

If further assistance is needed during insertion, as shown in FIG. 15, the clinician may further dynamically reduce the frictional force of inserting the device 280 into the hemostatic seal 1301 by reducing the axial compression to further dilate the hemostatic spring 851. For example, the clinician may apply additional force in direction 1401a to the trigger 1404 against the bias of compression spring 1313. In response to the additional application of force, the trigger 1404 again shifts in the direction 1401a to further compress the compression spring 1313 and further draw the hydraulic fluid 1321 into the compression chamber hydraulic cylinder 1319 from the compression chamber 1323. In response, the distal mount 1309 further shifts in the direction 1401b to further move the distal mount 1309 farther apart from the proximal mount 1303. Consequently, the hemostatic seal 1301 further dilates to further ease the frictional forces of inserting the device 280 while still providing sufficient constriction to seal against the surfaces of the device 280 to reduce fluid leakage from the introducer sheath 105 through the hemostatic seal 1301.

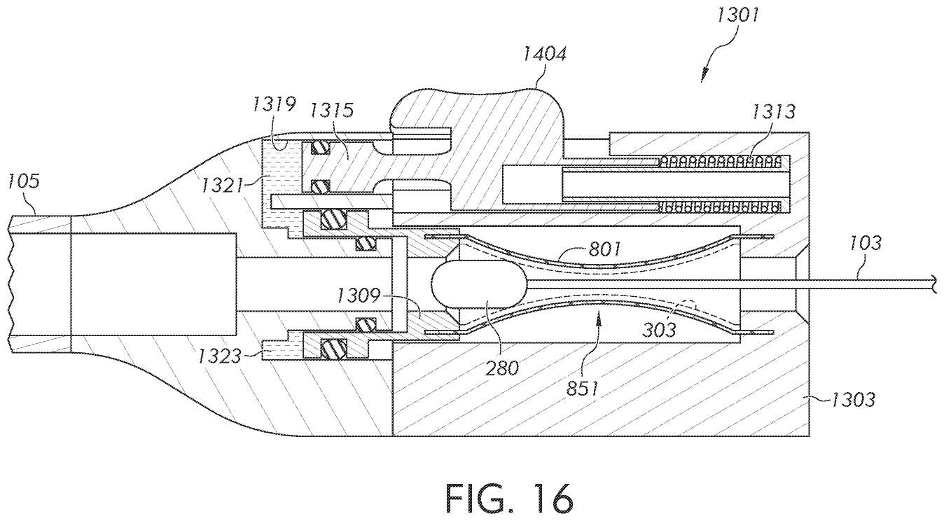

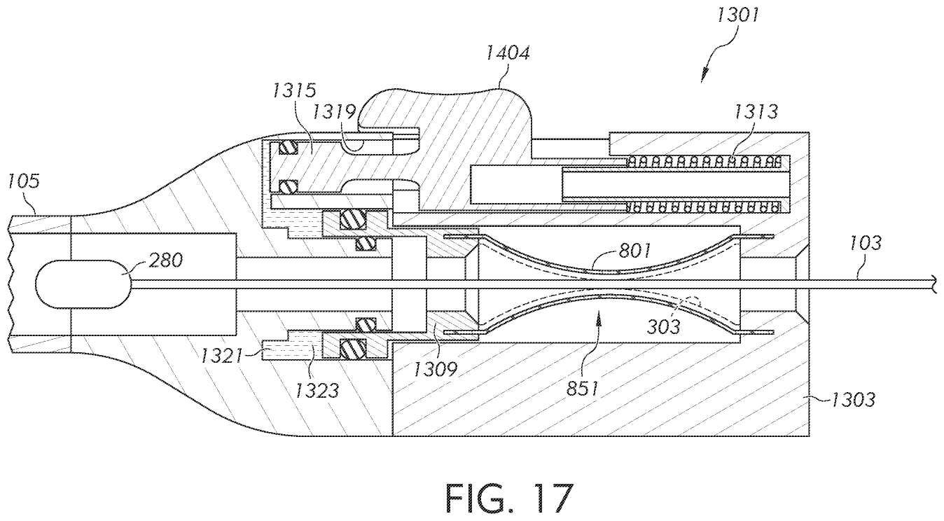

After reducing the frictional force, the method can subsequently include increasing the axial compression to constrict the hemostatic spring 851. For example, FIG. 15 represents the maximum device passage dimension that the hemostatic spring 851 achieves when the device 280 passes through the hemostatic seal 1301. Once the device passes, as illustrated in FIG. 16, the clinician may optionally release the trigger 1404. Once released, the compression spring 1313 will automatically apply the appropriate pressure to the hydraulic fluid 1321 to maintain the proper seal against the device 280 with the hemostatic spring 851. Indeed, as shown in FIG. 16, a clinician can insert the device 280 past the maximum device passage dimension and thereafter release the trigger 1404. The compression spring 1313 can then generate hydraulic pressure to maintain the axial compression of the hemostatic spring 251. Indeed, the compression spring 1313 compresses the hydraulic fluid 1321 within the hydraulic cylinder 1319 with the linear piston 1315. The hydraulic fluid 1321 then presses against the distal mount 1309 by entering the compression chamber 1323. The pressurized hydraulic fluid 1321 therefore biases the distal mount 1309 toward the proximal mount 1303 to bias the hemostatic spring 251 to constrict to maintain the axial compression in the hemostatic spring 251. As shown, the hemostatic spring 251 has constricted to seal against the device 280. As shown in FIG. 17, further biasing causes the hemostatic seal 1301 to seal against the exterior surfaces of the catheter 103 to further assist in reducing or preventing leaking of fluid from the introducer sheath 105. As further shown in FIG. 17, the method can comprise passing the device 280 into the introducer sheath 105 from the hemostatic seal 1301 and can then pass the device 280 from the introducer sheath 105 to the vasculature of a patient.

Methods of introducing a device 280 has been described with respect to the hemostatic seal 1301 with the understanding with similar or identical procedural steps can be performed with the other hemostatic seals 101, 1801, 2001 of the disclosure.

In some embodiments, methods of introducing the device 280 into the vasculature of a patient can comprise percutaneously inserting the introducer sheath 105 into the vasculature of the patient wherein the introducer sheath is provided with the hemostatic seal 101, 1301, 1801, 2001 comprising a hemostatic spring 251, 851, 1151, 1251 under axial compression to minimize leakage of fluid from the introducer sheath 105. The method can further comprise axially inserting the device 208 into the hemostatic seal 101, 1301, 1801, 2001. The method can further comprise reducing a frictional force of inserting the device 280 into the hemostatic seal 101, 1301, 1801, 2001 by reducing the axial compression to dilate the hemostatic spring 251, 851, 1151, 1251.

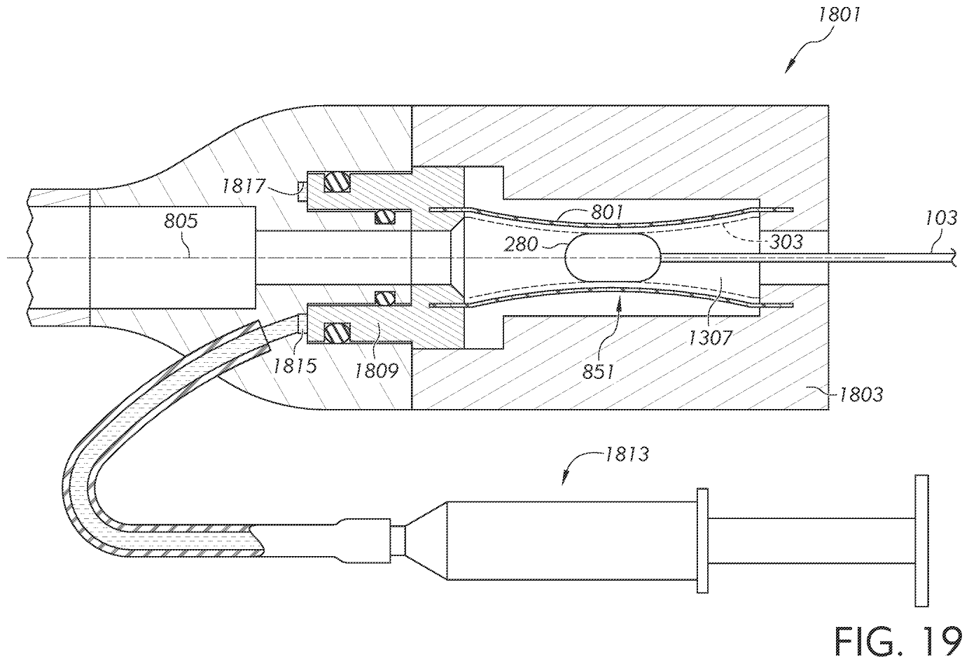

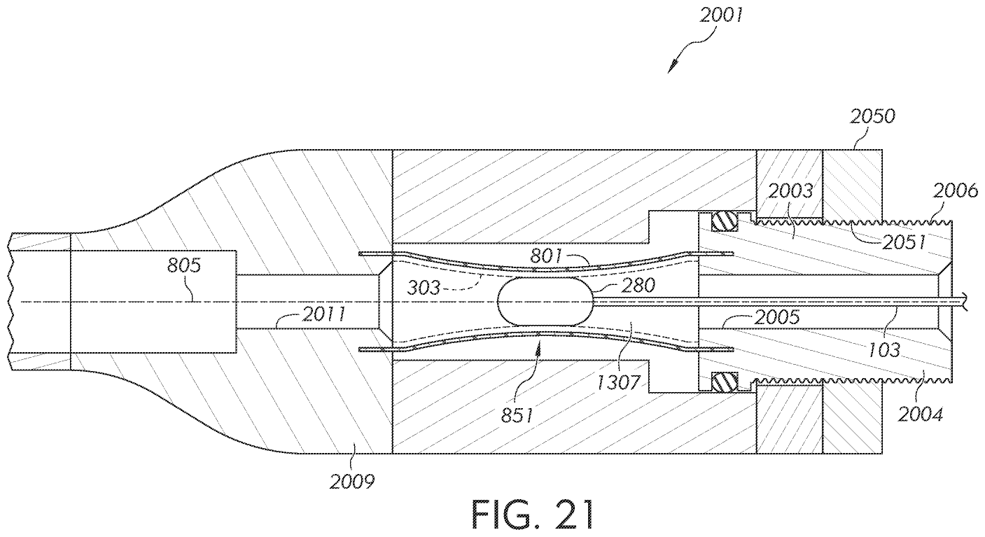

With initial schematical reference to FIG. 3, reducing the frictional force of inserting the device 280 into the hemostatic seal 101 can be conducted by reducing the axial compression to dilate the hemostatic spring 251 by increasing the length of the hemostatic spring 251 from L1 (see FIG. 2) to L2 (see FIG. 3). With reference to FIG. 14, reducing the frictional force of inserting the device 280 into the hemostatic seal 1301 can be conducted by reducing the axial compression to dilate the hemostatic spring 851 by moving the trigger 1404 in direction 1401a to increasing the length of the hemostatic spring 851 while compressing the compression spring 1313. With reference to FIG. 19, reducing the frictional force of inserting the device 280 into the hemostatic seal 1801 can be conducted by reducing the axial compression to dilate the hemostatic spring 851 by drawing hydraulic fluid into the syringe 1813 to increasing the length of the hemostatic spring 851. With reference to FIG. 21, reducing the frictional force of inserting the device 280 into the hemostatic seal 2001 can be conducted by reducing the axial compression to dilate the hemostatic spring 851 by rotating the drive nut 2050 to increase the length of the hemostatic spring 851. Reducing the frictional force can begin at least prior to the hemostatic spring 251, 851, 1151, 1251 dilating to a maximum device passage dimension (see FIGS. 3, 15, 19, and 21) that the hemostatic spring 251, 851, 1151, 1251 achieves when the device 280 passes through the hemostatic seal 101, 1301, 1801, 2001.

After reducing the frictional force, the method can comprise increasing the axial compression of the hemostatic spring to constrict the hemostatic spring. For example, in some embodiments, increasing the axial compression can occur after dilating the hemostatic spring to a maximum device passage dimension that the hemostatic spring achieves when the device passes through the hemostatic seal. For example, as shown in FIG. 16, the trigger 1404 can be released wherein force can be applied from the compression spring 1313 to maintain the axial compression of the hemostatic spring 851. Indeed, the compression spring 1313 can transfer force to compress the hydraulic fluid 1321. The compressed hydraulic fluid 1321 can thereafter force the distal mount 1309 closer to the proximal mount 1303 to maintain axial compression on the hemostatic spring 851. Referring to FIG. 18, in further embodiments, the syringe 1813 may force pressurized hydraulic fluid to apply pressure to the distal mount 1809 to maintain axial compression on the hemostatic spring. Hydraulic fluid throughout the disclosure can comprise a wide range of incompressible fluids such as water, saline, oil, or other fluids. Referring to FIG. 20, in further embodiments, the drive nut 2050 may be rotated to reduce the distance between the proximal mount 2003 and the distal mount 2009 to maintain axial compression on the hemostatic spring 851.

The methods of the disclosure can conclude by passing the device 280 into the introducer sheath 105 from the hemostatic seal 101, 1301, 1801, 2001 and then passing the device 280 from the introducer sheath 105 to the vasculature of the patient.

In accordance with the disclosure, non-limiting aspects of the disclosure will now be described. Various combinations of the aspects can be provided in accordance with the disclosure.

Aspect 1. A hemostatic spring comprising a first end portion, a second end portion opposite the first end portion, and a radial array of spring segments positioned on a circular path about a central axis. Each spring segment of the radial array of spring segments comprises a first end, a second end, and a central location positioned between the first end and the second end. The first end of each spring segment is attached to the first end portion and each spring segment tapers toward the central location in a first direction of the central axis extending from the first end portion toward the second end portion. A second end of each spring segment is attached to the second end portion and each spring segment tapers toward the central location in a second direction of the central axis opposite the first direction. The central location of each spring segment is spaced on the circular path from the central location of each adjacent spring segment of the radial array of spring segments when a force is not applied to the radial array of spring segments.

Aspect 2. The hemostatic spring of Aspect 1, wherein each spring segment continuously tapers in the first direction from the first end of the corresponding spring segment toward the central location of the corresponding spring segment. Each spring segment further continuously tapers in the second direction from the second end of the corresponding spring segment toward the central location of the corresponding spring segment.

Aspect 3. The hemostatic spring of any one of Aspects 1-2, wherein the central location of each spring segment comprises a midpoint of the corresponding spring segment.

Aspect 4. The hemostatic spring of any one of Aspects 1-2, wherein the central location of each spring segment comprises a central segment of the corresponding spring segment.

Aspect 5. The hemostatic spring of Aspect 4, wherein the central segment comprises a width that is substantially constant along the central axis.

Aspect 6. The hemostatic spring of Aspect 4, wherein each spring segment continuously tapers from the first end to a first central waist of the central location. Each spring segment further continuously tapers from the second end to a second central waist of the central location. The central segment is positioned between the first central waist and the second central waist.

Aspect 7. The hemostatic spring of Aspect 6, wherein a width of the central segment is greater than a width of the first central waist and a width of the second central waist.

Aspect 8. The hemostatic spring of Aspect 7, wherein the width of the central segment is substantially constant along the central axis.

Aspect 9. The hemostatic spring of Aspect 1, wherein the first end of each spring segment is attached at a first end waist to the first end portion and the second end of each spring segment is attached at a second end waist to the second end portion. Each spring segment tapers in the first direction from a first intermediate portion toward the central location of the corresponding spring segment, and each spring segment tapers in the second direction of the central axis from a second intermediate portion toward the central location of the corresponding spring segment. The first end waist is positioned between the first end portion and the first intermediate portion, and the second end waist is positioned between the second end portion and the second intermediate portion. The first intermediate portion comprises a width greater than a width of the first end waist, and the second intermediate portion comprises a width greater than a width of the second end waist.

Aspect 10. The hemostatic spring of Aspect 9, wherein a width of the first end waist and the width of the second end waist are each greater than a width of the central location.

Aspect 11. The hemostatic spring of Aspect 9, wherein the central location of each spring segment comprises a central segment of the corresponding spring segment.

Aspect 12. The hemostatic spring of Aspect 11, wherein the central segment comprises a width that is substantially constant along the central axis.

Aspect 13. The hemostatic spring of Aspect 12, wherein a width of the first end waist and the width of the second end waist are each greater than the width of the central segment.

Aspect 14. The hemostatic spring of any one of Aspects 9-13, wherein each spring segment continuously tapers in the first direction from the first intermediate portion to the central location of the corresponding spring segment. Each spring segment further continuously tapers in the second direction from the second end intermediate portion to the central location of the corresponding spring segment.

Aspect 15. The hemostatic spring of any one of Aspects 1-14, wherein each spring segment of the radial array of spring segments is substantially straight and extends parallel to the central axis when a force is not applied to the radial array of spring segments.

Aspect 16. The hemostatic spring of any one of Aspects 1-15, wherein the first end portion and the second end portion each comprise a continuous circular cylindrical tube.

Aspect 17. The hemostatic spring of any one of Aspects 1-16, wherein a thickness of the first end portion, a thickness of the second end portion, and a thickness of each spring segment are approximately equal.

Aspect 18. The hemostatic spring of any one of Aspects 1-17, further comprising a sleeve positioned within an interior of the hemostatic spring.

Aspect 19. The hemostatic spring of Aspect 18, wherein the sleeve comprises an elastomeric layer.

Aspect 20. The hemostatic spring of any one of Aspects 18-19, wherein the sleeve comprises a polymer layer.

Aspect 21. A hemostatic seal comprising a hemostatic spring comprising a first end portion, a second end portion opposite the first end portion, and a radial array of spring segments positioned on a circular path about a central axis. Each spring segment of the radial array of spring segments comprises a first end, a second end, and a central location positioned between the first end and the second end. The first end of each spring segment is attached to the first end portion and each spring segment tapers toward the central location in a first direction of the central axis extending from the first end portion toward the second end portion. A second end of each spring segment is attached to the second end portion and each spring segment tapers toward the central location in a second direction of the central axis opposite the first direction. The central location of each spring segment is spaced on the circular path from the central location of each adjacent spring segment of the radial array of spring segments when a force is not applied to the radial array of spring segments. The hemostatic seal further comprises a proximal mount comprising a proximal aperture aligned with the central axis extending through a central passage of the hemostatic spring, wherein the first end portion of the hemostatic spring is mounted to the proximal mount. The hemostatic seal further comprises a distal mount comprising a distal aperture aligned with the central axis, wherein the second end portion of the hemostatic spring is mounted to the distal mount. The hemostatic seal is configured to reduce the distance between the proximal mount and the distal mount along the central axis to constrict the radial array of spring elements to reduce a cross-sectional area of the central passage.

Aspect 22. The hemostatic seal of Aspect 21, further comprising a spring biasing at least one of the proximal mount and the distal mount to reduce the distance between the proximal mount and the distal mount along the central axis to constrict the radial array of spring elements.

Aspect 23. The hemostatic seal of Aspect 22, further comprising an actuator configured to act against the bias of the spring to dilate the radial array of spring segments to increase a cross-sectional area of the central passage.

Aspect 24. The hemostatic seal of Aspect 23, wherein the actuator comprises a linear trigger.

Aspect 25. The hemostatic seal of any one of Aspects 23-24, wherein the actuator comprises a linear piston.

Aspect 26. The hemostatic seal of Aspect 21, further comprising a fluid that is hydraulically compressed to bias at least one of the proximal mount and the distal mount to reduce the distance between the proximal mount and the distal mount along the central axis to constrict the radial array of spring elements.

Aspect 27. The hemostatic seal of any one of Aspects 21-26, wherein each spring segment continuously tapers in the first direction from the first end of the corresponding spring segment toward the central location of the corresponding spring segment. Each spring segment further continuously tapers in the second direction from the second end of the corresponding spring segment toward the central location of the corresponding spring segment.

Aspect 28. The hemostatic seal of any one of Aspects 21-27, wherein the central location of each spring segment comprises a midpoint of the corresponding spring segment.

Aspect 29. The hemostatic seal of any one of Aspects 21-27, wherein the central location of each spring segment comprises a central segment of the corresponding spring segment.

Aspect 30. The hemostatic seal of Aspect 29, wherein the central segment comprises a width that is substantially constant along the central axis.

Aspect 31. The hemostatic seal of Aspect 29, wherein each spring segment continuously tapers from the first end to a first central waist of the central location. Each spring segment further continuously tapers from the second end to a second central waist of the central location. The central segment is positioned between the first central waist and the second central waist.

Aspect 32. The hemostatic seal of Aspect 31, wherein the width of the central segment is greater than a width of the first central waist and a width of the second central waist.

Aspect 33. The hemostatic seal of Aspect 32, wherein the width of the central segment is substantially constant along the central axis.

Aspect 34. The hemostatic seal of any one of Aspects 21-26, wherein the first end of each spring segment is attached at a first end waist to the first end portion and the second end of each spring segment is attached at a second end waist to the second end portion. Each spring segment tapers in the first direction from a first intermediate portion toward the central location of the corresponding spring segment. Each spring segment further tapers in the second direction of the central axis from a second intermediate portion toward the central location of the corresponding spring segment. The first end waist is positioned between the first end portion and the first intermediate portion. The second end waist is positioned between the second end portion and the second intermediate portion. The first intermediate portion comprises a width greater than a width of the first end waist, and the second intermediate portion comprises a width greater than a width of the second end waist.

Aspect 35. The hemostatic seal of Aspect 34, wherein a width of the first end waist and the width of the second end waist are each greater than a width of the central location.

Aspect 36. The hemostatic seal of Aspect 34, wherein the central location of each spring segment comprises a central segment of the corresponding spring segment.

Aspect 37. The hemostatic seal of Aspect 36, wherein the central segment comprises a width that is substantially constant along the central axis.

Aspect 38. The hemostatic seal of Aspect 37, wherein a width of the first end waist and the width of the second end waist are each greater than the width of the central segment.

Aspect 39. The hemostatic seal of any one of Aspects 34-38, wherein each spring segment continuously tapers in the first direction from the first intermediate portion to the central location of the corresponding spring segment, and each spring continuously tapers in the second direction from the second end intermediate portion to the central location of the corresponding spring segment.

Aspect 40. The hemostatic seal of any one of Aspects 21-39, wherein each spring segment of the radial array of spring segments is substantially straight and extends parallel to the central axis when a force is not applied to the radial array of spring segments.

Aspect 41. The hemostatic seal of any one of Aspects 21-40, wherein the first end portion and the second end portion each comprise a continuous circular cylindrical tube.

Aspect 42. The hemostatic seal of any one of Aspects 21-41, wherein a thickness of the first end portion, a thickness of the second end portion, and a thickness of each spring segment are approximately equal.

Aspect 43. The hemostatic seal of any one of Aspects 21-42, further comprising a sleeve positioned within an interior of the hemostatic spring.

Aspect 44. The hemostatic seal of Aspect 43, wherein the sleeve comprises an elastomeric layer.

Aspect 45. The hemostatic seal of any one of Aspects 43-44, wherein the sleeve comprises a polymer layer.

Aspect 46. A method of introducing a device into a vasculature of a patient with the hemostatic seal of Aspect 21, comprising percutaneously inserting an introducer sheath into the vasculature of the patient, wherein the introducer sheath is in communication with the central passage, and the hemostatic spring is under an axial compression to minimize leakage of fluid from the introducer sheath. The method further comprises axially inserting the device into the hemostatic seal and reducing a frictional force of inserting the device into the hemostatic seal by reducing the axial compression to dilate the hemostatic spring. The method further comprises passing the device into the introducer sheath from the hemostatic seal, and passing the device from the introducer sheath to the vasculature of the patient.

Aspect 47. The method of Aspect 46, wherein, after reducing the frictional force, increasing the axial compression to constrict the hemostatic spring.

Aspect 48. The method of Aspect 47, wherein increasing the axial compression occurs after dilating the hemostatic spring to a maximum device passage dimension that the hemostatic spring achieves when the device passes through the hemostatic seal.

Aspect 49. The method of Aspect 46, wherein reducing the frictional force begins at least prior to the hemostatic spring dilating to a maximum device passage dimension that the hemostatic spring achieves when the device passes through the hemostatic seal.

Aspect 50. The method of Aspect 49, wherein after reducing the frictional force, reapplying the axial compression to constrict the hemostatic spring.

Aspect 51. The method of Aspect 50, wherein reapplying the axial compression occurs after dilating the hemostatic spring to the maximum device passage dimension.

Aspect 52. The method of any one of Aspects 46-51, further comprising applying force from a compression spring to maintain the axial compression of the hemostatic spring.

Aspect 53. The method of Aspect 52, wherein reducing the frictional force of inserting the device into the hemostatic seal comprises applying a force to compress the compression spring.

Aspect 54. The method of any one of Aspects 52-53, wherein the compression spring generates hydraulic pressure to maintain the axial compression of the hemostatic spring.

Aspect 55. The method of any one of Aspects 46-51, further comprising applying hydraulic pressure to maintain the axial compression in the hemostatic spring.

Aspect 56. The method of Aspect 55, wherein the hydraulic pressure is maintained by a syringe.

Aspect 57. The method of anyone of Aspects 46-56, wherein the hemostatic spring is under the axial compression to minimize the leakage of fluid from the introducer sheath by biasing at least one of the proximal mount and the distal mount to reduce a distance between the proximal mount and the distal mount along the central axis to constrict the radial array of spring elements.

Aspect 58. The method of Aspect 57, further comprising acting against the bias of the at least one of the proximal mount and the distal mount to dilate the radial array of spring segments to increase a cross-sectional area of the central passage.

Aspect 59. A method of introducing a device into a vasculature of a patient comprising percutaneously inserting an introducer sheath into the vasculature of the patient wherein the introducer sheath is provided with a hemostatic seal comprising a hemostatic spring under an axial compression to minimize leakage of fluid from the introducer sheath. The method further comprises axially inserting the device into the hemostatic seal, and reducing a frictional force of inserting the device into the hemostatic seal by reducing the axial compression to dilate the hemostatic spring. The method further comprises passing the device into the introducer sheath from the hemostatic seal and passing the device from the introducer sheath to the vasculature of the patient.

Aspect 60. The method of Aspect 59, wherein, after reducing the frictional force, increasing the axial compression to constrict the hemostatic spring.

Aspect 61. The method of Aspect 60, wherein increasing the axial compression occurs after dilating the hemostatic spring to a maximum device passage dimension that the hemostatic spring achieves when the device passes through the hemostatic seal.

Aspect 62. The method of Aspect 59, wherein reducing the frictional force begins at least prior to the hemostatic spring dilating to a maximum device passage dimension that the hemostatic spring achieves when the device passes through the hemostatic seal.

Aspect 63. The method of Aspect 62, wherein after reducing the frictional force, reapplying the axial compression to constrict the hemostatic spring.

Aspect 64. The method of Aspect 63, wherein reapplying the axial compression occurs after dilating the hemostatic spring to the maximum device passage dimension.

Aspect 65. The method of any one of Aspects 59-64, further comprising applying force from a compression spring to maintain the axial compression of the hemostatic spring.

Aspect 66. The method of Aspect 65, wherein reducing the frictional force of inserting the device into the hemostatic seal comprises applying a force to compress the compression spring.

Aspect 67. The method of any one of Aspects 65-66, wherein the compression spring generates hydraulic pressure to maintain the axial compression of the hemostatic spring.

Aspect 68. The method of any one of Aspects 59-64, further comprising applying hydraulic pressure to maintain the axial compression in the hemostatic spring.

Aspect 69. The method of Aspect 68, wherein the hydraulic pressure is maintained by a syringe.

It should be understood that while various aspects have been described in detail relative to certain illustrative and specific examples thereof, the present disclosure should not be considered limited to such, as numerous modifications and combinations of the disclosed features are possible without departing from the scope of the following claims.

What is claimed is:

1. A hemostatic spring comprising:
a first end portion;
a second end portion opposite the first end portion;
a radial array of spring segments positioned on a circular path about a central axis, each spring segment of the radial array of spring segments comprising a first end, a second end, and a central location positioned between the first end and the second end, wherein the first end of each spring segment is attached to the first end portion and each spring segment tapers toward the central location in a first direction of the central axis extending from the first end portion toward the second end portion, and a second end of each spring segment is attached to the second end portion and each spring segment tapers toward the central location in a second direction of the central axis opposite the first direction,
wherein the central location of each spring segment is spaced on the circular path from the central location of each adjacent spring segment of the radial array of spring segments when a force is not applied to the radial array of spring segments.

2. The hemostatic spring of claim 1, wherein each spring segment continuously tapers in the first direction from the first end of the corresponding spring segment toward the central location of the corresponding spring segment, and each spring segment continuously tapers in the second direction from the second end of the corresponding spring segment toward the central location of the corresponding spring segment.

3. The hemostatic spring of claim 1, wherein the central location of each spring segment comprises a midpoint of the corresponding spring segment.

4. The hemostatic spring of claim 1, wherein the central location of each spring segment comprises a central segment of the corresponding spring segment.

5. The hemostatic spring of claim 4, wherein the central segment comprises a width that is substantially constant along the central axis.

6. The hemostatic spring of claim 4, wherein each spring segment continuously tapers from the first end to a first central waist of the central location, and continuously tapers from the second end to a second central waist of the central location, wherein the central segment is positioned between the first central waist and the second central waist.

7. The hemostatic spring of claim 6, wherein a width of the central segment is greater than a width of the first central waist and a width of the second central waist.

8. The hemostatic spring of claim 7, wherein the width of the central segment is substantially constant along the central axis.

9. The hemostatic spring of claim 1, wherein the first end of each spring segment is attached at a first end waist to the first end portion and the second end of each spring segment is attached at a second end waist to the second end portion, wherein the each spring segment tapers in the first direction from a first intermediate portion toward the central location of the corresponding spring segment, and each spring segment tapers in the second direction of the central axis from a second intermediate portion toward the central location of the corresponding spring segment, wherein the first end waist is positioned between the first end portion and the first intermediate portion, the second end waist is positioned between the second end portion and the second intermediate portion, the first intermediate portion comprises a width greater than a width of the first end waist, and the second intermediate portion comprises a width greater than a width of the second end waist.

10. The hemostatic spring of claim 9, wherein a width of the first end waist and the width of the second end waist are each greater than a width of the central location.

11. The hemostatic spring of claim 9, wherein the central location of each spring segment comprises a central segment of the corresponding spring segment.

12. The hemostatic spring of claim 11, wherein the central segment comprises a width that is substantially constant along the central axis.

13. The hemostatic spring of claim 12, wherein a width of the first end waist and the width of the second end waist are each greater than the width of the central segment.

14. The hemostatic spring of claim 9, wherein each spring segment continuously tapers in the first direction from the first intermediate portion to the central location of the corresponding spring segment, and each spring continuously tapers in the second direction from the second end intermediate portion to the central location of the corresponding spring segment.

15. The hemostatic spring of claim 1, wherein each spring segment of the radial array of spring segments is substantially straight and extends parallel to the central axis when a force is not applied to the radial array of spring segments.

16. The hemostatic spring of claim 1, wherein the first end portion and the second end portion each comprise a continuous circular cylindrical tube.

17. The hemostatic spring of claim 1, wherein a thickness of the first end portion, a thickness of the second end portion, and a thickness of each spring segment are approximately equal.

18. The hemostatic spring of claim 1, further comprising a sleeve positioned within an interior of the hemostatic spring.

19. The hemostatic spring of claim 18, wherein the sleeve comprises an elastomeric layer.

20. The hemostatic spring of claim 18, wherein the sleeve comprises a polymer layer.
